(19) United States
(12) Patent Application Publication
(10) Pub. No.: US 2026/0112476 A1
(43) Pub. Date: Apr. 23, 2026

(54) WORK ASSISTANCE APPARATUS AND NON-TRANSITORY COMPUTER-READABLE RECORDING MEDIUM STORING WORK ASSISTANCE PROGRAM

(71) Applicant: Konica Minolta, Inc., Tokyo (JP)

(72) Inventor: Sumiya NAGATSUKA, Tokyo (JP)

(21) Appl. No.: 19/357,789

(22) Filed: Oct. 14, 2025

(30) Foreign Application Priority Data
Oct. 21, 2024 (JP) 2024-185015

(51) Int. Cl. G16H 30/20; G16H 30/40; G16H 50/20

(52) U.S. Cl. CPC G16H 30/20; G16H 30/40; G16H 50/20

(57) ABSTRACT

Provided is a work assistance apparatus including: a display that displays an analysis image for a dynamic image to be processed, which has been obtained by irradiating a subject with radiation; and at least one hardware processor. The at least one hardware processor executes dialogue including reception of an input of a question about the displayed analysis image and an output of a response to the inputted question, and outputs case information corresponding to the dynamic image to be processed, based on the displayed analysis image and a content of the executed dialogue.

### CROSS REFERENCE TO RELATED APPLICATIONS

The entire disclosure of Japanese Patent Application No. 2024-185015, filed on October 21, 2024, is incorporated herein by reference in its entirety.

### Background

### Technological Field

The present invention relates to a work assistance apparatus and a non-transitory computer-readable recording medium storing a work assistance program.

### Description of Related Art

There is known an assistance apparatus that assists a medical doctor in diagnosis by analyzing, with a computer, a dynamic image obtained by irradiating a patient with radiation. For example, the apparatus described in Japanese Patent Application Laid-Open No. 2004-000410 assists a medical doctor in diagnosis by marking a disease-suspicious site on a still image by image analysis, marking a site corresponding to the disease-suspicious site on a dynamic image, and providing such still and dynamic images.

Diagnosis of a still image obtained by radiation irradiation is often performed by a specialized medical doctor called a radiologist. For this reason, even in a case where still and dynamic images as described above are provided, it is often burdensome and time-consuming for a medical doctor with little experience to perform diagnosis based on dynamic images. In addition, the diagnosis based on dynamic images is a relatively new diagnosis, and even a radiologist may take time for the diagnosis. For this reason, there is a demand for a work assistance apparatus capable of not only marking disease-suspicious sites, but also assisting in work related to diagnosis of dynamic images to improve efficiency in the work.

### SUMMARY

An object of the present invention is to provide a work assistance apparatus and a non-transitory computer-readable recording medium storing a work assistance program each capable of improving efficiency in work related to diagnosis of dynamic images.

In order to achieve at least one of the above-mentioned objects, a work assistance apparatus reflecting one aspect of the present invention includes:

a display that displays an analysis image for a dynamic image to be processed, which has been obtained by irradiating a subject with radiation; and

at least one hardware processor.

The at least one hardware processor

executes dialogue including reception of an input of a question about the displayed analysis image and an output of a response to the inputted question, and

outputs case information corresponding to the dynamic image to be processed, based on the displayed analysis image and a content of the executed dialogue.

In order to achieve at least one of the above-mentioned objects, a non-transitory computer-readable recording medium storing a work assistance program reflecting one aspect of the present invention is a non-transitory computer-readable recording medium storing a work assistance program that causes a computer of a work assistance apparatus, where the work assistance apparatus assists in case diagnosis of a dynamic image to be processed which has been obtained by irradiating a subject with radiation, to execute:

displaying an analysis image for the dynamic image to be processed;

executing dialogue including reception of an input of a question about the displayed analysis image and an output of a response to the inputted question; and

outputting case information corresponding to the dynamic image to be processed, based on the displayed analysis image and a content of the executed dialogue.

### BRIEF DESCRIPTION OF DRAWINGS

The advantages and features provided by one or more embodiments of the invention will become more fully understood from the detailed description given hereinbelow and the appended drawings which are given by way of illustration only, and thus are not intended as a definition of the limits of the present invention:

FIG. 1 is a diagram for describing an example of a configuration of a radiographic image processing system;

FIG. 2 is a block diagram for describing an example of a functional configuration of an imaging controller in a radiographic image capturing apparatus constituting the radiographic image processing system;

FIG. 3 is a block diagram for describing an example of a functional configuration of a radiographic imaging control apparatus constituting the radiographic image processing system;

FIG. 4 is a block diagram for describing an example of a functional configuration of a radiographic image analysis apparatus constituting the radiographic image processing system;

FIG. 5 is a block diagram for describing an example of a functional configuration of a client terminal that also serves as a work assistance apparatus according to an embodiment of the present invention;

FIG. 6 is a flowchart for describing a work assistance program (work assistance method) that is carried out at the client terminal;

FIG. 7 is a diagram illustrating an example of a list of dynamic images displayed on a display;

FIG. 8 is a diagram illustrating an exemplary analysis image and an exemplary menu for operation which are displayed on the display;

FIG. 9 is a diagram illustrating an exemplary analysis image and an exemplary dialogue region which are displayed on the display, illustrating an exemplary question inputted into the dialogue region;

FIG. 10 is a diagram illustrating an exemplary answer to the question inputted in FIG. 9, illustrating the exemplary answer displayed in the dialogue region and exemplary analysis images similar to an analysis image displayed on the display;

FIG. 11 is a diagram illustrating an exemplary newly inputted question and an answer to the new question, which are displayed on the display; and

FIG. 12 is a diagram illustrating an exemplary analysis image, exemplary analysis images similar to the analysis image, and an exemplary menu for operation, which are displayed on the display.

### DETAILED DESCRIPTION OF EMBODIMENTS

Hereinafter, one or more embodiments of the present invention will be described with reference to the drawings. However, the scope of the invention is not limited to the disclosed embodiments.

Hereinafter, an embodiment of the present invention will be described in detail with reference to the accompanying drawings.

### Configuration of Radiation Image Processing System

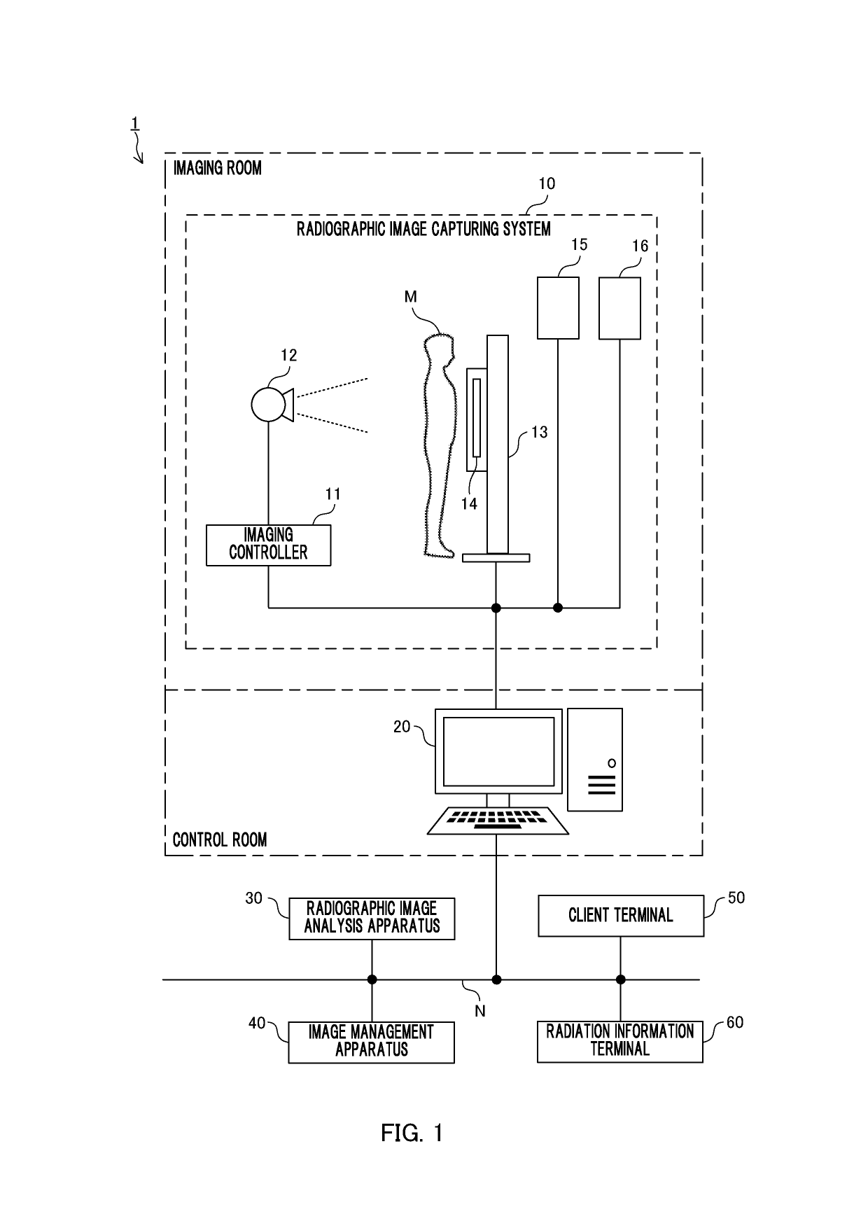

FIG. 1 is a diagram for describing a radiographic image processing system 1 in the present embodiment. The radiographic image processing system 1 includes a radiographic image capturing system 10, a radiographic imaging control apparatus (console apparatus) 20, a radiographic image analysis apparatus 30, an image management apparatus 40, and a client terminal 50.

In the example illustrated in FIG. 1, the radiographic image capturing system 10 is disposed in an imaging room, and the radiographic imaging control apparatus 20 is disposed in a control room. The radiographic image capturing system 10, the radiographic imaging control apparatus 20, the radiographic image analysis apparatus 30, the image management apparatus 40, and the client terminal 50 are connected to each other via a communication network N. As the communication network N, a communication network compliant with, for example, the Digital Image and Communications in Medicine (DICOM) standard or the like is used.

In addition, the communication network N is connected to a radiation information terminal 60 serving as a radiation information system that transmits information on a radiation inspection, such as inspection order information on a patient, to the radiographic image processing system 1. The radiation information terminal 60 is, for example, a radiology information system (RIS) or the like.

The radiographic image capturing system 10 performs radiographic dynamic imaging (hereinafter referred to as dynamic imaging), in which a radiographic dynamic image (hereinafter referred to as a dynamic image) is captured, based on the control of the radiographic imaging control apparatus 20. The radiographic imaging control apparatus 20 controls the radiographic image capturing system 10 based on inspection order information or the like transmitted from the radiation information terminal 60. A dynamic image generated by the radiographic image capturing system 10 is subjected to processing at the radiographic imaging control apparatus 20, which will be described later, and is transmitted to the radiographic image analysis apparatus 30. The radiographic image analysis apparatus 30 executes dynamic analysis on a dynamic image. A dynamic image and a dynamic analysis result are transmitted to and managed by the image management apparatus 40 as a medical image management system. The image management apparatus 40 is, for example, a picture archiving and communication system (PACS) or the like. The dynamic image and the dynamic analysis result are transmitted to the client terminal 50 and are viewed by a medical worker such as a medical doctor.

In the present embodiment, the dynamic imaging refers to obtaining a plurality of frame images by repeatedly irradiating a subject with pulsed radiation (for example, X-rays) at a predetermined frame rate (pulse irradiation). In addition, the dynamic image refers to a series of frame images obtained by the dynamic imaging. In addition, the dynamic analysis refers to analysis processing performed on a dynamic image, and includes, in addition to processing of analyzing the movement of a subject based on the dynamic image, processing of analyzing the dynamic image to highlight or attenuate (remove) a predetermined structure.

Each of the radiographic image capturing system 10, the radiographic imaging control apparatus 20, and the radiographic image analysis apparatus 30 is a kind of computer that includes a processor and a memory, and implements a predetermined function by reading, developing, and executing a program stored in the memory.

### Radiographic Image Capturing System 10

As illustrated in FIG. 1, the radiographic image capturing system 10 includes an imaging controller 11, a radiation irradiator 12, an imaging table 13, a radiation detector 14, a display 15, and a sound outputter 16.

The imaging controller 11 acquires setting information on settings of dynamic imaging from the radiographic imaging control apparatus 20. The imaging controller 11 sets imaging conditions for performing dynamic imaging based on the setting information, and controls the radiation irradiator 12 based on the imaging conditions to cause a patient M (subject) to be irradiated with radiation and imaging to be performed. The imaging controller 11 is constituted by a central processing unit (CPU), a random access memory (RAM), a read only memory (ROM), and the like.

The setting information is information on settings for performing dynamic imaging on the patient M. The setting information includes, for example, at least one of a plurality of types of dynamic analyses that can be executed on a dynamic image by the radiographic image analysis apparatus 30. In a case where a plurality of types of dynamic analyses is combined, the setting information may include information on the combination. In the radiographic imaging control apparatus 20 to be described later, the setting information is set by the operational person of the radiographic image processing system 1, for example, an imaging technician or the like.

The imaging conditions include, for example, various conditions such as a pulse rate, a pulse width, a pulse interval, the number of imaging frames per imaging, a dose per unit time of radiation irradiation, an imaging posture of the patient M, and the body state (respiratory state or the like) of the patient M. The pulse rate is the number of times of radiation irradiation per second, and coincides with the frame rate of image data. The pulse width is a radiation irradiation time per radiation irradiation. The pulse interval is a time from the start of one radiation irradiation to the start of the next radiation irradiation, and coincides with a time interval (frame interval) between a plurality of image data. The imaging conditions may be automatically determined by the imaging controller 11 of the radiographic image capturing system 10 based on the setting information.

The radiation irradiator 12 is disposed at a position facing the radiation detector 14 fixed to the imaging table 13. The radiation irradiator 12 irradiates radiation according to the control of the imaging controller 11.

The radiation detector 14 is constituted by a semiconductor image sensor, such as a flat panel detector (FPD). The radiation detector 14 includes a board in which a plurality of detection elements (pixels) is arranged in a matrix, where the plurality of detection elements (pixels) detects the radiation irradiated by the radiation irradiator 12 according to the intensity of the radiation, converts the detected radiation into an electrical signal, and accumulate the electrical signal. Each pixel of the board is configured to include, for example, a switcher such as a thin film transistor (TFT).

The radiation detector 14 controls the switcher of each pixel based on image reading conditions inputted through the radiographic imaging control apparatus 20 to read electrical signals accumulated in each pixel, and outputs intensity information for each pixel to an image generator 113. The image reading conditions are, for example, a frame rate, a frame interval, a pixel size, an image size (matrix size), and the like. The frame rate is the number of frame images acquired per second, and coincides with the pulse rate. The frame interval is a time from the start of one operation of acquiring image data to the start of the operation of acquiring the next frame image, and coincides with the pulse interval.

The imaging controller 11 and the radiation detector 14 are connected to each other, and are configured to exchange synchronization signals with each other to synchronize the radiation irradiation operation with the image reading operation.

As described above, the radiographic image capturing system 10 performs dynamic imaging of a radiographic image by the radiation irradiator 12 irradiating radiation and the radiation detector 14 generating image data based on the intensity of the irradiated radiation under the control of the controller 11.

The display 15 and the sound outputter 16 provide the patient M with instructions on a posture to be taken (imaging posture), a body state, a respiratory state, and the like when dynamic imaging of the patient M is performed. The display 15 is, for example, a display apparatus such as a cathode ray tube (CRT), a liquid crystal display, or an organic electro luminescence (EL) display. The sound outputter 16 is, for example, a sound output apparatus such as a speaker. The sound outputter 16 provides the patient M with instructions on the body state, the respiratory state, and the like by, for example, automatic voice. Each of the display 15 and the sound outputter 16 may provide the patient M with instructions having the same content to the patient M, or only one of the display 15 and the sound outputter 16 may provide the patient M with instructions.

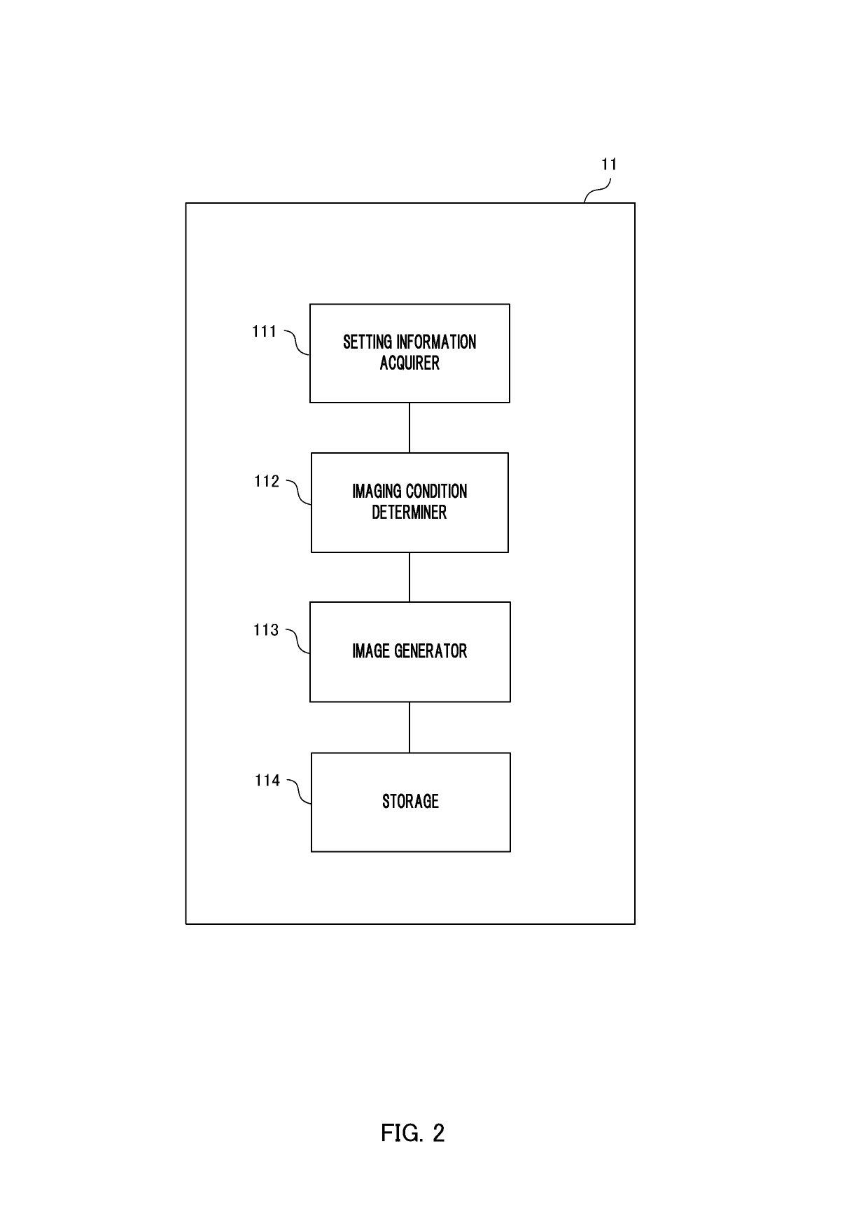

FIG. 2 is a block diagram for describing an example of a functional configuration of the imaging controller 11 in the radiographic image capturing system 10 constituting the radiographic image processing system 1. The imaging controller 11 includes a setting information acquirer 111, an imaging condition determiner 112, the image generator 113, and a storage 114.

The setting information acquirer 111 acquires setting information from the radiographic imaging control apparatus 20.

The imaging condition determiner 112 determines imaging conditions when dynamic imaging of the patient M is performed, based on the setting information. Information indicating correspondence relationships between a plurality of types of dynamic analyses and imaging conditions suitable for the respective dynamic analyses is stored in the storage 114 in advance. In addition, information indicating a correspondence relationship between a combination of a plurality of types of dynamic analyses and imaging conditions suitable for the combination is also stored in the storage 114 in advance. With respect to the dynamic analysis indicated by the setting information or the combination of the plurality of types of dynamic analyses, the imaging condition determiner 112 may determine the imaging conditions by reading the information indicating the correspondence relationship from the storage 114 and collating the information with the setting information.

Note that, for example, in the case of screening, emergency, or the like, the dynamic analysis serving as the setting information may not be set. In such a case, the imaging condition determiner 112 determines the imaging condition(s) by causing the operational person to select at least one imaging condition from a plurality of predefined imaging conditions. In addition, the imaging condition determiner 112 causes the operational person to select inspection order information and determines the imaging conditions based on the selected inspection order information. As described above, in a case where the dynamic analysis cannot be set before the dynamic imaging, the dynamic analysis is set after the dynamic imaging under the imaging condition(s) selected by the operational person, and the dynamic analysis at the radiographic image analysis apparatus 30 to be described later (analysis in an analyzer 312 to be described later) is executed.

The image generator 113 executes dynamic imaging on the patient M based on the determined imaging condition(s) and generates a radiographic image with a plurality of frames. Specifically, the image generator 113 controls the operations of the radiation irradiator 12 and the radiation detector 14 based on the imaging condition(s), and generates image data by acquiring, for each pixel, intensity information on the intensity of radiation transmitted through the subject from the radiation detector 14.

As described above, the storage 114 stores, in advance, the information indicating the correspondence relationships between the plurality of types of dynamic analyses and the imaging conditions suitable for the respective dynamic analyses, the information indicating the correspondence relationship between the combination of the plurality of types of dynamic analyses and the imaging conditions suitable for the combination, and/or the like.

### Radiographic Imaging Control Apparatus 20

The radiographic imaging control apparatus 20 is, for example, a computer such as a personal computer (PC) or a workstation. The radiographic imaging control apparatus 20 may be a desktop computer as in the example illustrated in FIG. 1, or may be a portable computer, such as a notebook computer or a tablet computer, or the like.

The radiographic imaging control apparatus 20 controls dynamic imaging of the radiographic image capturing system 10 by receiving inspection order information from the radiation information terminal 60 or the like and transmitting the inspection order information to the radiographic image capturing system 10.

The inspection order information includes various information on dynamic imaging to be executed next, such as instruction information on respiration and imaging posture, patient information, inspection information, imaging information, and data attributes. The inspection information includes information such as an inspection ID, an inspection target site (for example, the chest, in particular the lungs, the heart, or the like), and types of analyses (for example, ventilation analysis, pulmonary blood flow analysis, measurement of the maximal ventilation volume, and the like). The inspection order information is generated, for example, in a case where a medical doctor or the like requests the radiographic image processing system 1 to perform dynamic imaging of the patient M, or the like.

In addition, the radiographic imaging control apparatus 20 generates setting information indicating at least one dynamic analysis among a plurality of types of dynamic analyses executable by the radiographic image analysis apparatus 30 based on an input by the operational person. In the case of combining a plurality of types of dynamic analyses, the radiographic imaging control apparatus 20 generates setting information indicating the combination of the plurality of types of dynamic analyses. The operational person recognizes which dynamic analyses are to be combined among the plurality of types of dynamic analyses by referring to, for example, the content of the inspection order information and performs an input operation for generating the setting information based on the recognition. Alternatively, the operational person may also recognize which dynamic analyses are to be combined, based on information transmitted from a medical doctor or the like by another method.

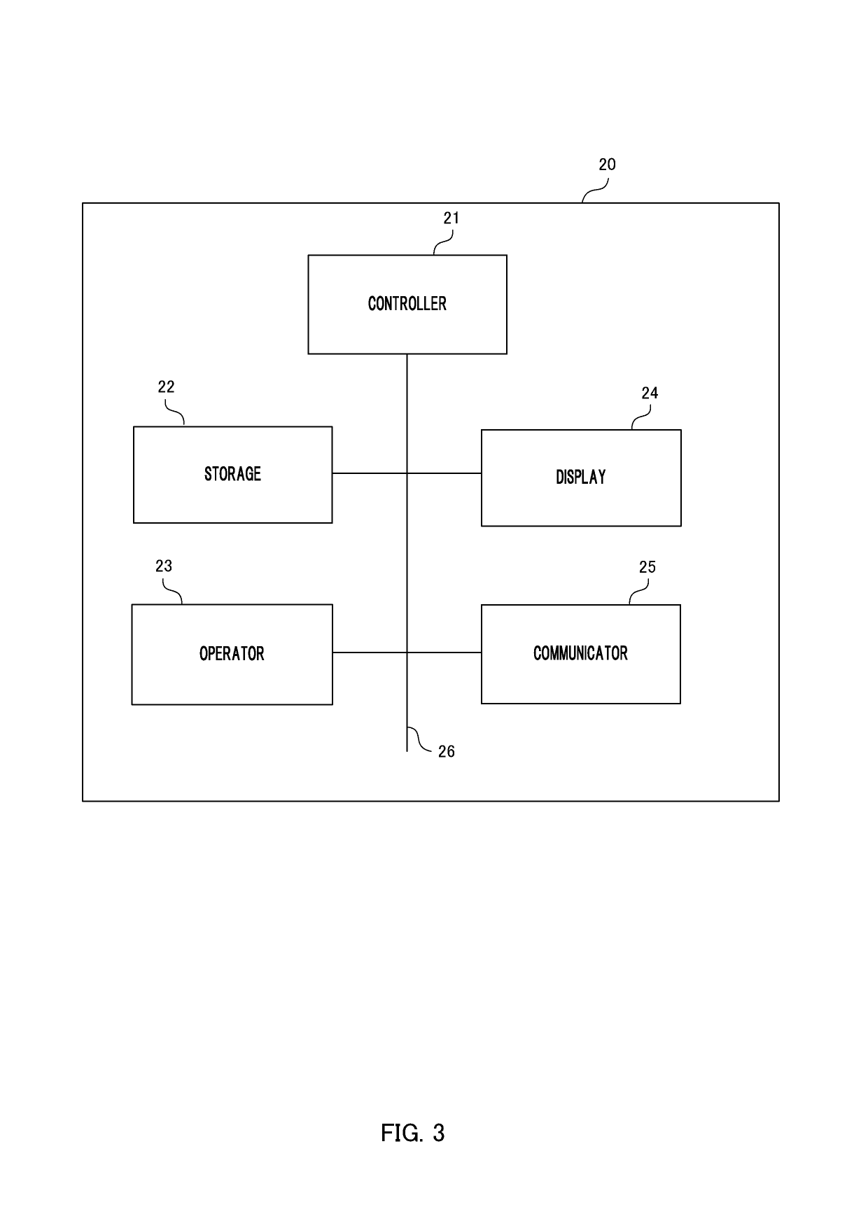

FIG. 3 is a block diagram for describing an example of a functional configuration of the radiographic imaging control apparatus 20 constituting the radiographic image processing system 1. The radiographic imaging control apparatus 20 includes a controller 21, a storage 22, an operator 23, a display 24, and a communicator 25. The respective configurations included in the radiographic imaging control apparatus 20 are connected to each other via a bus 26.

The radiographic imaging control apparatus 20 outputs, to the radiographic image capturing system 10, setting conditions set by the operational person or the like and inspection order information acquired in advance from the radiation information terminal 60 or the like, and controls imaging processing by the radiographic image capturing system 10. The radiographic imaging control apparatus 20 may display a dynamic image generated by the radiographic image capturing system 10, for example, for the operational person to check the dynamic image.

The controller 21 is constituted by a CPU, a RAM, and the like. In the controller 21, in response to an operation of the operator 23, the CPU reads a system program and various processing programs stored in the storage 22, develops the system program and various processing programs in the RAM, and controls the operation of each unit of the radiographic imaging control apparatus 20 based on the developed programs.

The storage 22 is constituted by a non-volatile semiconductor memory, a hard disk, or the like. The storage 22 stores various programs to be executed by the controller 21, parameters required for execution of processing by the programs, or data such as processing results (a dynamic image or the like). The various programs are stored in the form of readable program codes, and the controller 21 sequentially executes operations according to the program codes.

In addition, the storage 22 stores image reading conditions for performing dynamic imaging. Further, the storage 22 stores inspection order information transmitted from the radiation information terminal 60 or the like. When the radiographic imaging control apparatus 20 controls the dynamic imaging of the radiographic image capturing system 10, the radiographic imaging control apparatus 20 reads the image reading conditions and the inspection order information, which correspond to the patient M, from the storage 22 and transmits the image reading conditions and the inspection order information.

The operator 23 is an operation device such as a keyboard including a cursor key, number input keys, various function keys and the like, a pointing device such as a mouse or a trackball, a touch screen, and the like. The operator 23 generates an instruction signal based on an input by the operational person, and outputs the instruction signal to the controller 21.

The display 24 is constituted by a display device such as a CRT, a liquid crystal display, or an organic EL display. The display 24 displays an input instruction from the operator 23, image data (a dynamic image or the like) generated by the radiographic image capturing system 10, and the like according to an instruction of a display signal inputted from the controller 21.

The communicator 25 transmits and receives data to and from the radiographic image capturing system 10, the radiographic image analysis apparatus 30, the radiation information terminal 60, and the like.

### Radiographic Image Analysis Apparatus 30

The radiographic image analysis apparatus 30 is, for example, a computer such as a PC or a workstation. The radiographic image analysis apparatus 30 may be a desktop computer, or may be a portable computer, such as a notebook computer or a tablet computer, or the like.

The radiographic image analysis apparatus 30 executes dynamic analysis on a dynamic image captured by the radiographic image capturing system 10 based on the setting information set at the radiographic imaging control apparatus 20.

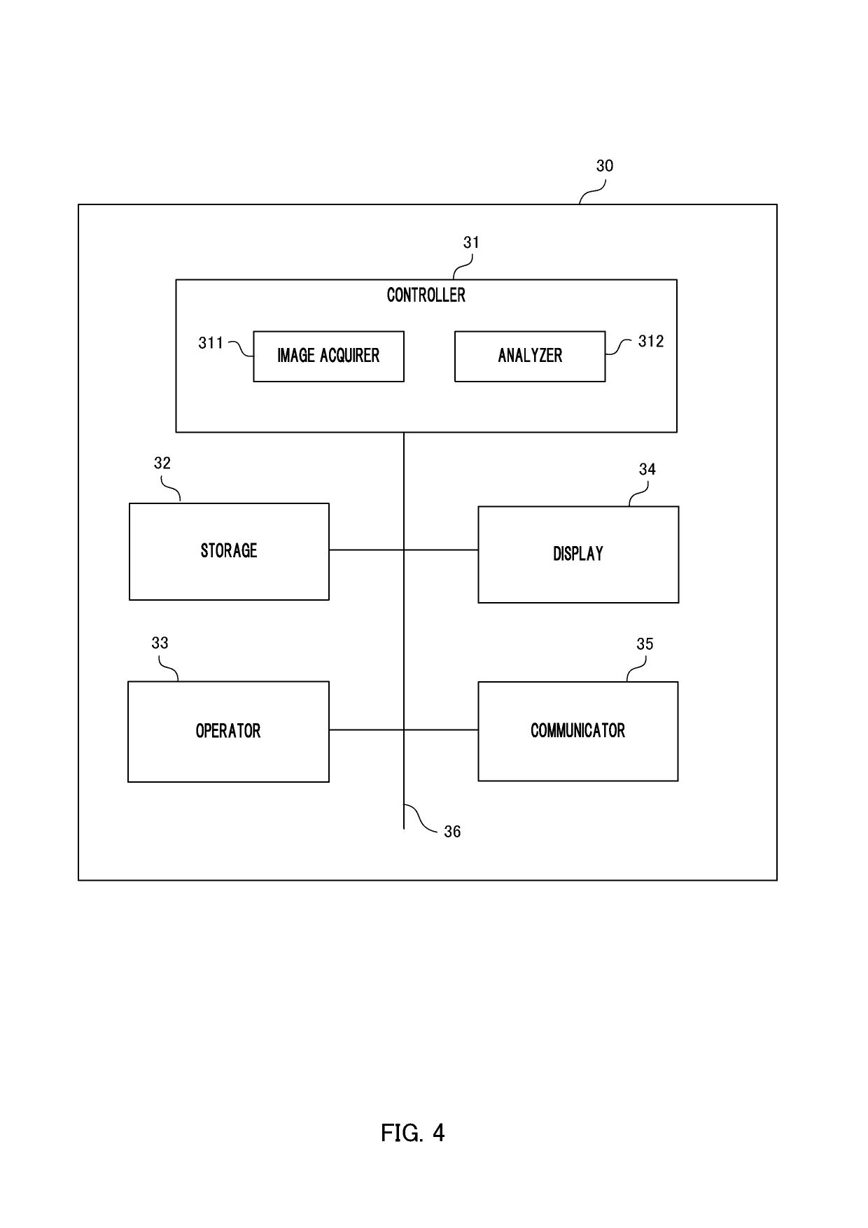

FIG. 4 is a block diagram for describing an example of a functional configuration of the radiographic image analysis apparatus 30 constituting the radiographic image processing system 1. The radiographic image analysis apparatus 30 includes a controller 31, a storage 32, an operator 33, a display 34, and a communicator 35. The respective configurations included in the radiographic image analysis apparatus 30 are connected to each other by a bus 36.

The controller 31 is constituted by a CPU, a RAM, and the like. In the controller 31, in response to an operation of the operator 33, the CPU reads a system program and various processing programs stored in the storage 32, develops the system program and various processing programs in the RAM, and executes operation control of each unit of the radiographic image analysis apparatus 30, dynamic analysis, and the like based on the developed programs.

The controller 31 includes an image acquirer 311 and the analyzer 312.

The image acquirer 311 acquires a dynamic image that is a radiographic image with a plurality of frames generated by the radiographic image capturing system 10 and the radiographic imaging control apparatus 20.

The analyzer 312 executes the dynamic analysis set by the setting information on a dynamic image including a plurality of frame images, acquires an analysis result, and generates an analysis image for displaying the analysis result on the dynamic image. For example, the analyzer 312 performs analysis based on signal changes in a plurality of frame images and displays an analysis result based on the signal changes on the dynamic image. At this time, in a case where the analyzer 312 cannot analyze the dynamic image (cannot acquire the analysis result), the analyzer 312 determines that the analysis is not possible.

The analyzer 312 has, for example, a blood flow analysis mode, a ventilation analysis mode, an adhesion analysis mode, a diaphragm movement amount analysis mode, an orthopedic-related measurement mode, and the like as the types of dynamic analyses. Each mode will be briefly described below.

The blood flow analysis mode is a mode in which a signal change in the lung field synchronized with the heartbeat is visualized.

The ventilation analysis mode is a mode in which a signal change in a time direction in a specific time-frequency band is extracted and lung tissue behavior during respiration is visualized.

The adhesion analysis mode is a mode in which the degree of adhesion of tissues is visualized.

The diaphragm movement amount analysis mode is a mode in which the upward/downward movement of the diaphragm associated with respiration is tracked.

The orthopedic-related measurement mode is, for example, a mode in which a change in the position of a specified bone in the four limbs or the like is measured and the trajectory of the movement is displayed.

The storage 32 is constituted by a non-volatile semiconductor memory, a hard disk, or the like. The storage 32 stores various programs to be executed by the controller 31, parameters required for execution of processing by the programs, or data such as processing results (a dynamic image, an analysis result, an analysis image(s), and the like). The various programs are stored in the form of readable program codes, and the controller 31 sequentially executes operations in accordance with the program codes.

In addition, the storage 32 stores patient information, inspection information, and list information indicating the status (for example, the progress status such as during reception, during dynamic analysis, completion of analysis, and the like) according to each dynamic image generated by the radiographic image capturing system 10. Further, in the storage 32, analysis results and analysis images are stored in association with dynamic images.

The operator 33 is an operation device such as a keyboard including a cursor key, number input keys, and various function keys, a pointing device such as a mouse or a trackball, a touch screen, and the like. The operator 33 generates an instruction signal based on an input by the operational person, and outputs the instruction signal to the controller 31. In addition, the operator 33 may include a touch screen on the display screen of the display 34, and in this case, outputs an instruction signal inputted through the touch screen to the controller 31.

The display 34 is constituted by a display device such as a CRT, a liquid crystal display, or an organic EL display. The display 34 displays an input instruction from the operator 33, image data (a dynamic image, an analysis result, an analysis image(s), and the like) generated by the radiographic image capturing system 10, and the like according to an instruction of a display signal inputted from the controller 31.

The communicator 35 transmits and receives data to and from the radiographic imaging control apparatus 20, the image management apparatus 40, and the like.

### Client Terminal 50

The client terminal 50 (the work assistance apparatus according to the present invention) is, for example, a computer such as a PC or a workstation. The client terminal 50 may be a desktop computer or a portable computer, such as a notebook computer or a tablet computer, or the like.

The client terminal 50 is a computer used by a medical doctor. On the client terminal 50, the medical doctor views information such as a dynamic image, an analysis result, an analysis image(s) as described above. In the present embodiment, the client terminal 50 is configured to assist a medical doctor in diagnosis based on information such as a dynamic image, an analysis result, and an analysis image(s), as described later.

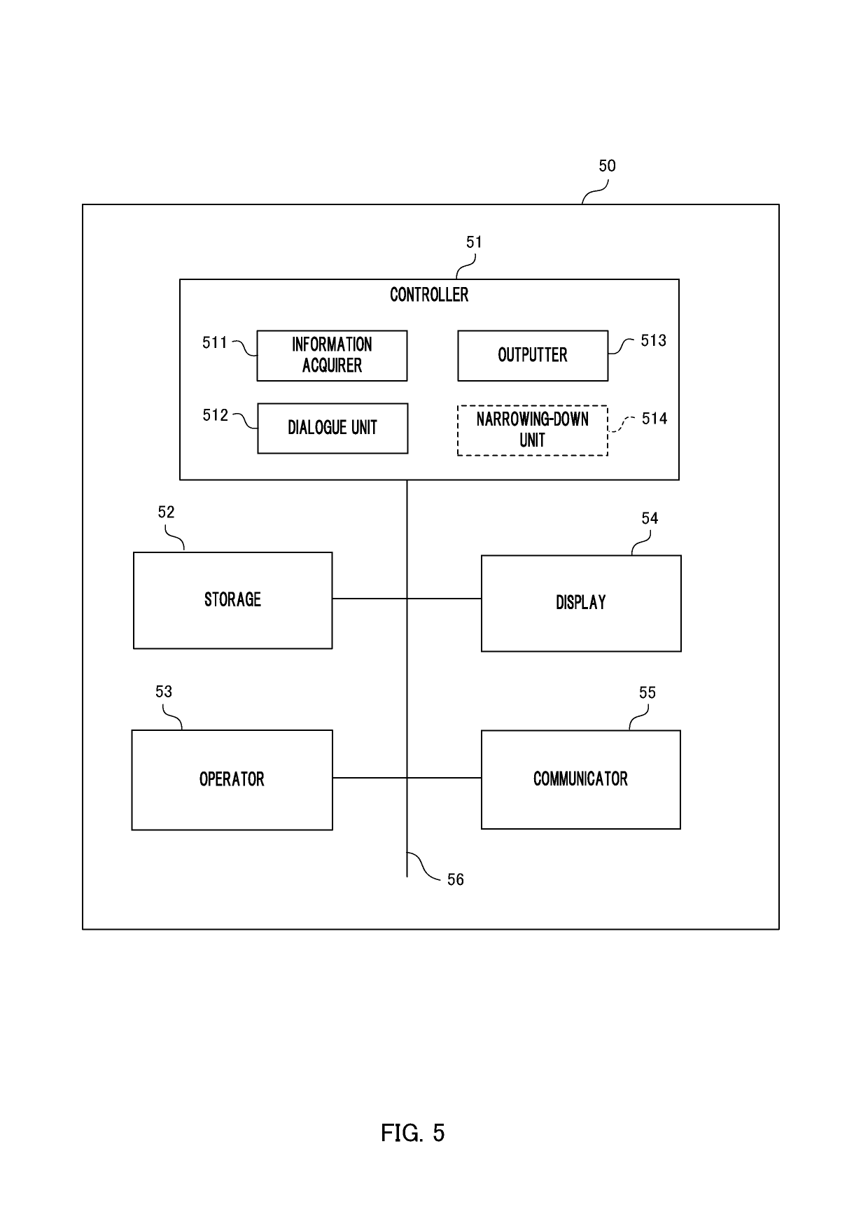

FIG. 5 is a block diagram for describing an example of a functional configuration of the client terminal 50 that also serves as a work assistance apparatus according to the present embodiment.

The client terminal 50 includes a controller 51, a storage 52, an operator 53, a display 54, and a communicator 55. The respective configurations included in the client terminal 50 are connected to each other by a bus 56.

The controller 51 is, for example, a computer including at least one hardware processor, and is constituted by a CPU, a RAM, and the like. In the controller 51, in response to an operation of the operator 53, the CPU reads a system program and various processing programs stored in the storage 52, develops the system program and various processing programs in the RAM, and performs processing for information viewing based on the developed programs. In the case of the present embodiment, the controller 51 executes, for example, a work assistance program, which will be described later, as a processing program, and provides information for assisting a medical doctor in diagnosis. The work assistance program is stored in a non-transitory computer-readable recording medium, and the work assistance program is stored in the storage 52 from the recording medium.

The controller 51 includes an information acquirer 511, a dialogue unit 512, and an outputter 513.

The information acquirer 511 acquires information, such as a dynamic image to be processed, an analysis result, and an analysis image(s), from the image management apparatus 40 based on an input operation by a medical doctor.

The dynamic image, the analysis result, the analysis image, and the like acquired by the information acquirer 511 are displayed on the display 54 and are provided to the medical doctor so that the medical doctor can view the dynamic image, the analysis result, the analysis image, and the like. Hereinafter, the analysis image will be described as an example of a target of displaying and a question, but the target thereof may be the dynamic image or the analysis result, or may be a plurality of or all of the dynamic image, the analysis result, and the analysis image.

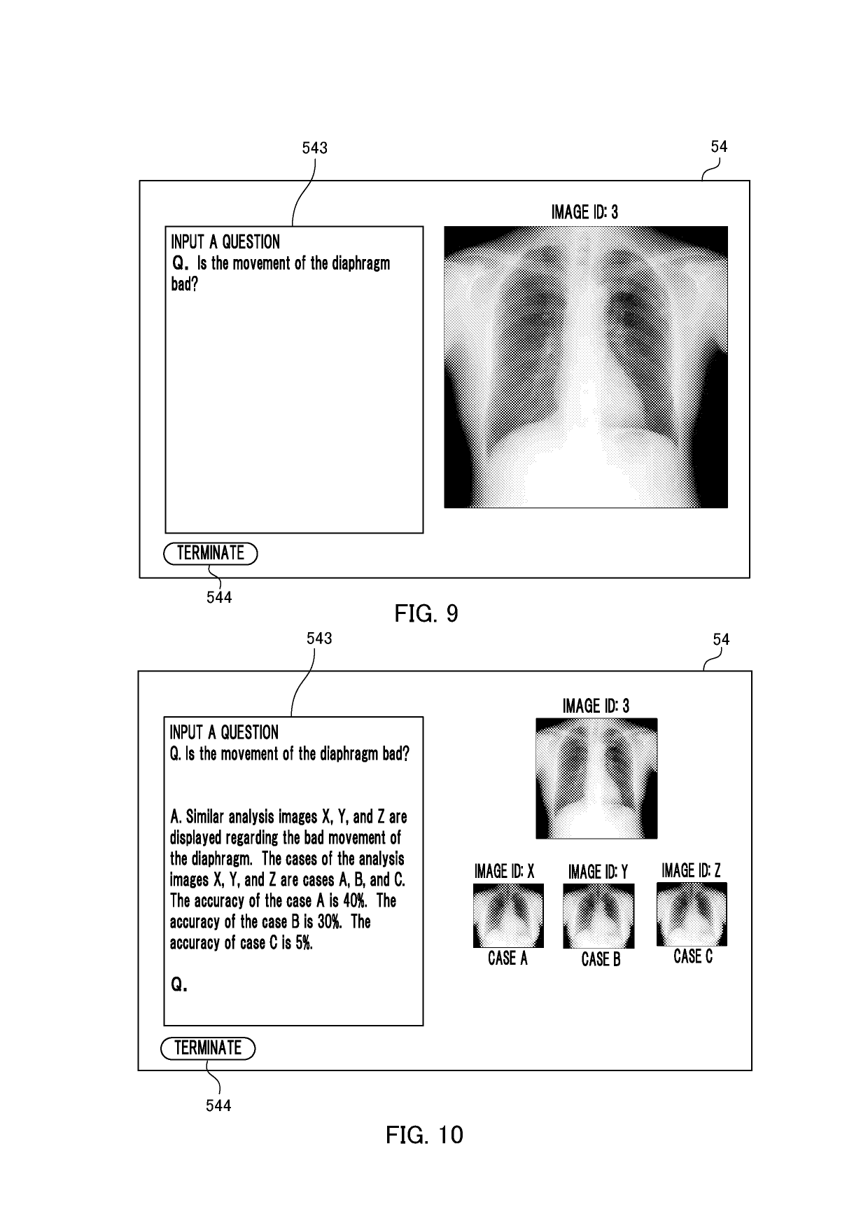

The dialogue unit 512 executes dialogue including reception of an input of a question about an analysis image, which is an analysis image for a dynamic image to be processed and is displayed on the display 54 (hereinafter referred to as an analysis image to be processed), and an output of a response to the inputted question. For example, as illustrated in FIG. 9 or the like to be described later, the dialogue unit 512 causes a dialogue region 543 to be displayed on the display 54 and performs the dialogue in the dialogue region 543.

The dialogue unit 512 may limit the number of times of dialogue to a predetermined number of times defined in advance, and in that case, processing by the outputter 513 is performed after dialogue has been performed the predetermined number of times. In a case where the number of times of questions is too large, the accuracy of retrieval or extraction is lowered depending on the contents of the questions, and the efficiency is also lowered. For this reason, for example, the predetermined number of times is limited to three times or the like.

In addition, the dialogue unit 512 may include a template for receiving an input of a question, and in that case, the medical doctor inputs a question in line with the template.

In addition, although illustration is omitted, the dialogue unit 512 includes a natural language processor, recognizing a natural language inputted into the dialogue region 543 to perform processing such that the natural language can be used for retrieval or extraction at the outputter 513. In addition, the dialogue unit 512 may include a sound recognition processor, and in this case, performs sound recognition of sound inputted through a sound inputter (for example, a microphone or the like) included in the client terminal 50, and inputs the sound into the dialogue region 543.

The outputter 513 retrieves and extracts an analysis image similar to the analysis image to be processed from first retrieval data (to be described later) stored in the storage 52 based on the analysis image to be processed and the content of the executed dialogue. Then, the outputter 513 outputs the dynamic image including the extracted analysis image, and case information thereof. In a case where the number of times of dialogue is limited to a predetermined number of times, the outputter 513 may extract an analysis image similar to the analysis image to be processed based on the analysis image to be processed and the content of the dialogue executed the predetermined number of times. In addition, the outputter 513 may extract a plurality of analysis images, and in that case, may output a plurality of case candidates as case information. At this time, the outputter 513 may output the plurality of case candidates in the order of accuracy with respect to the dynamic image to be processed.

In addition, although illustration is omitted, the outputter 513 includes a learner and an extractor. The learner generates a learning model by machine learning of the first retrieval data. The extractor retrieves and extracts an analysis image(s) similar to the analysis image to be processed from the first retrieval data by using the learning model with the analysis image to be processed and the content of the dialogue as input items. The input items are not limited to the analysis image and the content of the dialogue, and may include, for example, information such as imaging conditions.

In a case where the controller 51 does not include the dialogue unit 512, the outputter 513 retrieves and extracts an analysis image(s) similar to the analysis image to be processed from the first retrieval data based on the analysis image to be processed. For example, the outputter 513 retrieves and extracts an analysis image(s) similar to the analysis image to be processed from the first retrieval data by using the learning model with the analysis image to be processed as an input item. The learning model is good at providing a typical output based on a typical input, but it is difficult to obtain an output when an analysis image is atypical. For this reason, in the present embodiment, a similar analysis image(s) is/are retrieved and extracted in a dialogical manner by using the dialogue unit 512, so that an appropriate output can be obtained even in a case where the analysis image is atypical.

The storage 52 is constituted by a non-volatile semiconductor memory, a hard disk, or the like. The storage 52 stores various programs to be executed by the controller 51, parameters required for execution of processing by the programs, and the like. The various programs are stored in the form of readable program codes, and the controller 51 sequentially executes operations in accordance with the program codes.

In addition, the storage 52 stores first retrieval data including a plurality of dynamic images, accompanying information thereof (analysis images, imaging conditions, and the like), and a definite diagnosis result of a case based on each of the plurality of dynamic images. The first retrieval data is retrieved by the outputter 513 based on the analysis image to be processed and the content of the executed dialogue, and an analysis image(s) similar to the analysis image to be processed is/are extracted. With respect to the plurality of dynamic images, the storage 52 may update the first retrieval data based on the definite diagnosis result of the case based on the dynamic images, and a definite diagnosis result of a case based on an inspection other than an inspection using the dynamic images.

Note that, the first retrieval data may be included in the image management apparatus 40. In this case, the outputter 513 may retrieve and extract a similar analysis image(s) from the first retrieval data included in the image management apparatus 40, and output the case information of the dynamic image including the analysis image(s).

The operator 53 is an operation device such as a keyboard including a cursor key, number input keys, and various function keys, a pointing device such as a mouse or a trackball, a touch screen, and the like. The operator 53 generates an instruction signal based on an input by the operational person, and outputs the instruction signal to the controller 51. In addition, the operator 53 may include a touch screen on the display screen of the display 54, and in this case, outputs an instruction signal inputted through the touch screen to the controller 51.

The display 54 is constituted by a display device such as a CRT, a liquid crystal display, or an organic EL display. The display 54 displays an input instruction from the operator 53, a dynamic image, an analysis result, an analysis image(s), and the like according to an instruction of a display signal inputted from the controller 31.

The communicator 55 transmits and receives data to and from the image management apparatus 40 and the like.

### Work Assistance Program (Work Assistance Method)

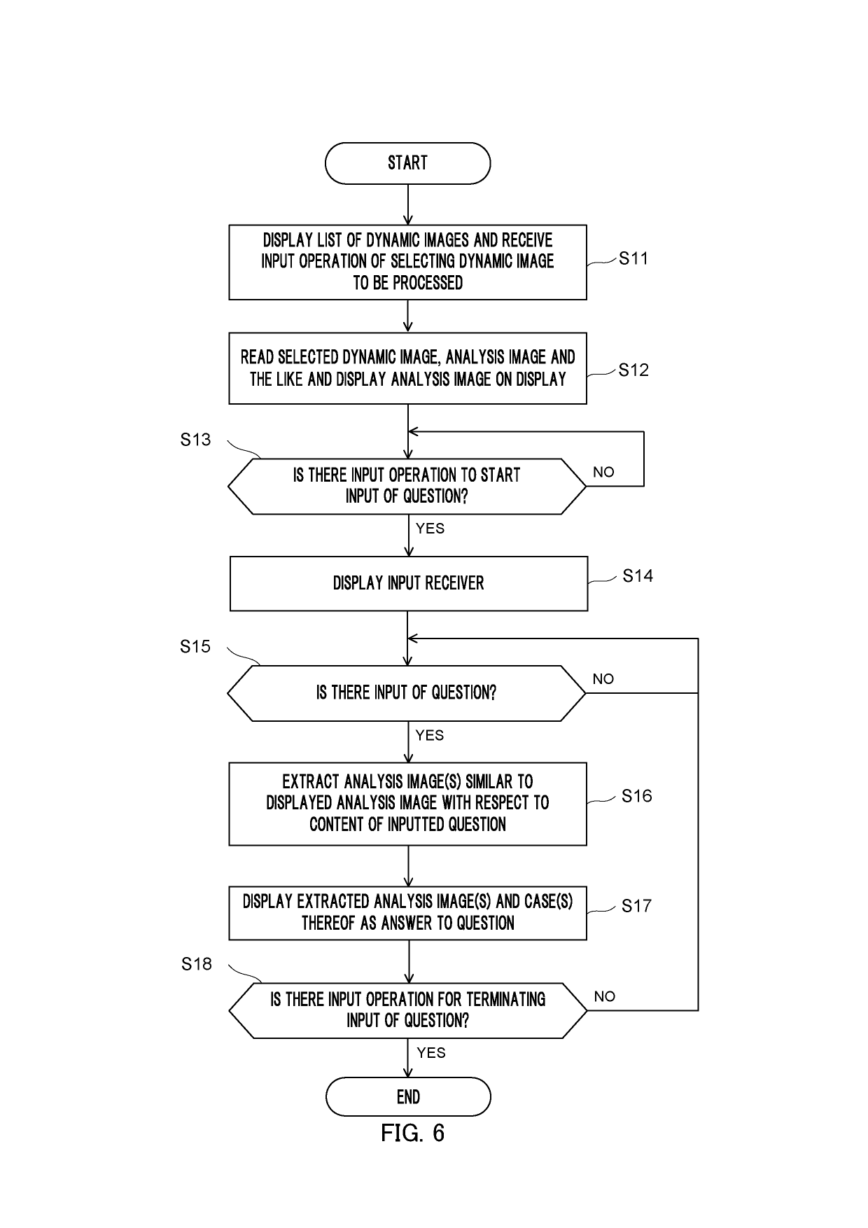

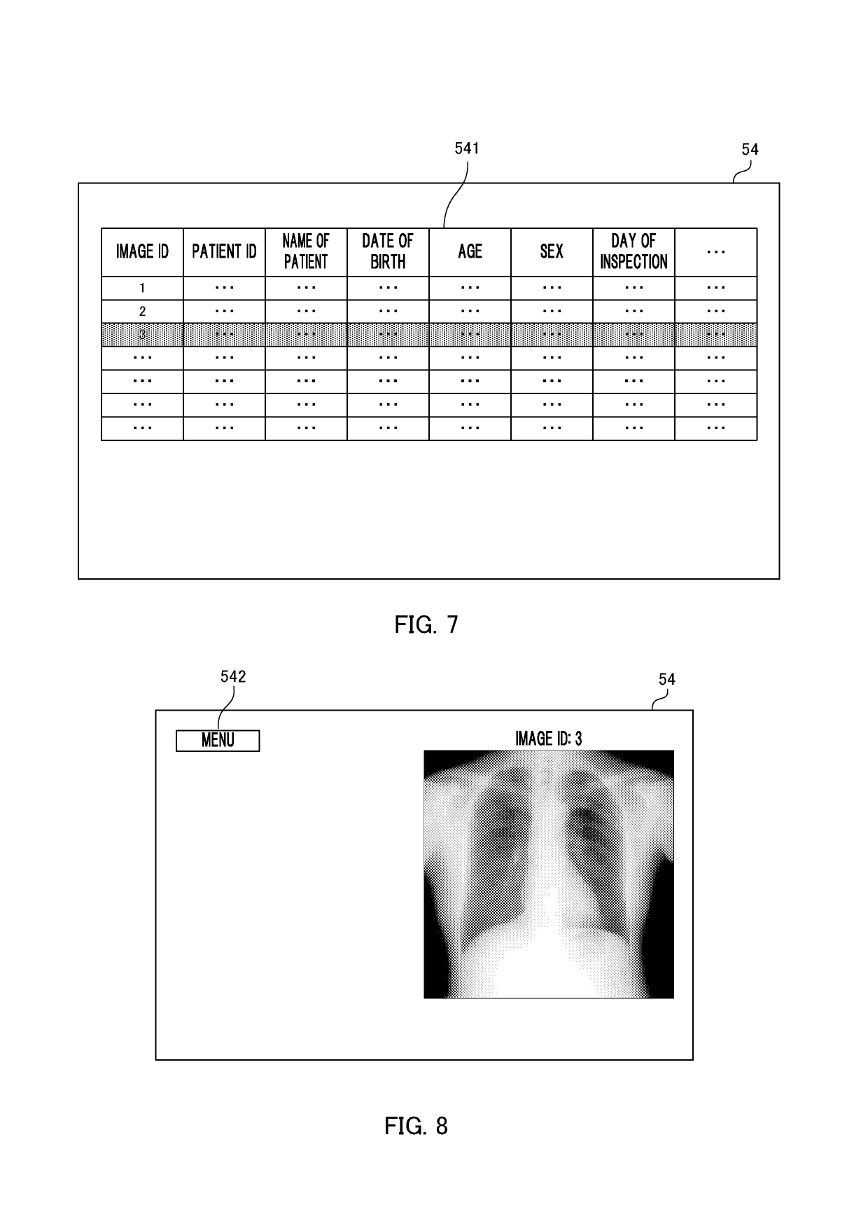

FIG. 6 is a flowchart for describing a work assistance program (work assistance method) that is carried out by the client terminal 50. FIGS. 7 to 11 are diagrams illustrating display examples on the display. Note that here, the description is given by taking, as an example, a case where the dynamic analysis of the movement of the diaphragm is performed in the above-described diaphragm movement amount analysis mode, but the following method is also applicable to cases where the dynamic analysis is performed in any of the above-described other analysis modes.

### Step S11

The controller 51 (the information acquirer 511) of the client terminal 50 acquires a list 541 of dynamic images to be processed from the image management apparatus 40 via the communicator 55 and the communication network N. The controller 51 causes the acquired list 541 of dynamic images to be displayed on the display 54. For example, as illustrated in FIG. 7, the controller 51 causes the list 541 including the image IDs, the patient IDs, and the like of the respective dynamic images to be displayed on the display 54. The controller 51 receives an input operation of selecting a dynamic image to be processed from the list 541 displayed on the display 54 via the operator 53.

For example, in FIG. 7, when it is assumed that the dynamic image of the image ID: 3 is the dynamic image to be processed, the medical doctor selects, for example, the dynamic image of the image ID: 3 from the list 541 displayed on the display 54 by using the mouse or the cursor key of the operator 53.

### Step S12

The controller 51 reads, together with the selected dynamic image of the image ID: 3, an analysis result and an analysis image from the image management apparatus 40 via the communicator 55 and the communication network N, and causes the analysis image of the image ID: 3 to be displayed on the display 54. The analysis image is reproduced as a moving image on the display 54, and the number of times of reproduction, the reproduction speed, and the like can be changed by an operation of the medical doctor who views the analysis image.

In addition, for example, as illustrated in FIG. 8, the controller 51 causes a menu 542 for operation together with the analysis image of the image ID: 3 to be displayed on the display 42. Note that, the analysis image illustrated in FIG. 8 and FIGS. 9 to 12 to be described later is an example in which a colored analysis image in one analysis mode is illustrated in black and white. In the present embodiment, analysis images having analysis results with various display forms are displayed on the display 42 according to the analysis modes.

When the menu 542 is selected, a plurality of input operation items is displayed on the display 54, and the medical doctor selects a desired item from among the plurality of input operation items. The plurality of input operation items includes an input operation item for starting an input of a question.

### Step S13

The controller 51 checks whether there is an input operation to start an input of a question via the operator 53. That is, the controller 51 checks whether the input operation item for starting an input of a question has been selected. In a case where there is an input operation to start (YES), the processing proceeds to step S14, and in a case where there is no input operation to start (NO), step S13 is repeated until there is an input operation to start.

### Step S14

The controller 51 causes the dialogue region 543 to be displayed on the display 54. For example, as illustrated in FIG. 9, the controller 51 causes the dialogue region 543 to be displayed in a region different from the region in which the analysis image of the image ID: 3 is displayed.

### Step S15

The controller 51 checks, via the operator 53, whether there is an input into the dialogue region 543. In a case where there is an input into the dialogue region 543 (YES), the processing proceeds to step S16. In a case where there is no input into the dialogue region 543 (NO), step S15 is repeated until there is an input into the dialogue region 543.

### Step S16

The controller 51 (the dialogue unit 512) retrieves and extracts, from the first retrieval data in the storage 52, an analysis image(s) similar to the analysis image of the image ID: 3 with respect to the content of the inputted question.

For example, for an analysis image that a medical doctor views, the medical doctor inputs, as the content of a question, what the medical doctor finds abnormal or wishes to check. For example, as illustrated in FIG. 9, it is assumed that the content of a question is “Is the movement of the diaphragm bad?”. In this case, the controller 51 retrieves and extracts an analysis image(s) similar to the analysis image of the image ID: 3 from the first retrieval data in the storage 52.

### Step S17

The controller 51 (the outputter 513) reads the extracted analysis image(s) and the case(s) in a dynamic image(s) including the analysis image(s) from the first retrieval data in the storage 52, and causes the read extracted analysis image(s) and case(s) to be displayed on the display 54 as an answer to the question.

For example, it is assumed that there is a plurality of analysis images (for example, images ID: X, ID: Y, and ID: Z) that is similar to the analysis image of the image ID: 3 in terms of similarity in the bad movement of the diaphragm. In this case, as illustrated in FIG. 10, the outputter 513 displays, together with the analysis images of the plurality of images ID: X, ID: Y, and ID: Z, the cases thereof (for example, a case A, a case B, and a case C) on the display 54.

In addition, as illustrated in FIG. 10, the outputter 513 displays a response in the form of a text to the inputted question in the dialogue region 543. The text serving as the response includes, for example, the image IDs, candidates for the cases, the accuracy of each case, and the like. For example, in FIG. 10, the text is that “Similar analysis images X, Y, and Z are displayed regarding the bad movement of the diaphragm. The cases of the analysis images X, Y, and Z are cases A, B, and C. The accuracy of the case A is 40%. The accuracy of the case B is 30%. The accuracy of case C is 5%.”

### Step S18

The controller 51 checks whether there is an input operation for terminating the input of the question via the operator 53. For example, the controller 51 checks whether a termination region 544 illustrated in FIGS. 9 and 10 is selected. In a case where there is an input operation to terminate (YES), the above processing is terminated, and in a case where there is no input operation to terminate (NO), the processing returns to step S15.

In a case where the processing returns to step S15 and a new question is inputted, the controller 51 repeats the above-described steps S16 and S17 with respect to the content of the newly inputted question. In this case, in a case where a predefined term, for example, “cancellation of extraction” or the like is included in the content of the new question, the controller 51 cancels the dynamic image(s) extracted hitherto, and performs the retrieval and extraction of the first retrieval data based on the new question inputted together with “cancellation of extraction”.

In a case where the defined term such as “cancellation of extraction” is not included in the content of the new question, on the other hand, the controller 51 repeats the above-described steps S16 and S17 with respect to the content of the new question based on the extracted analysis image(s).

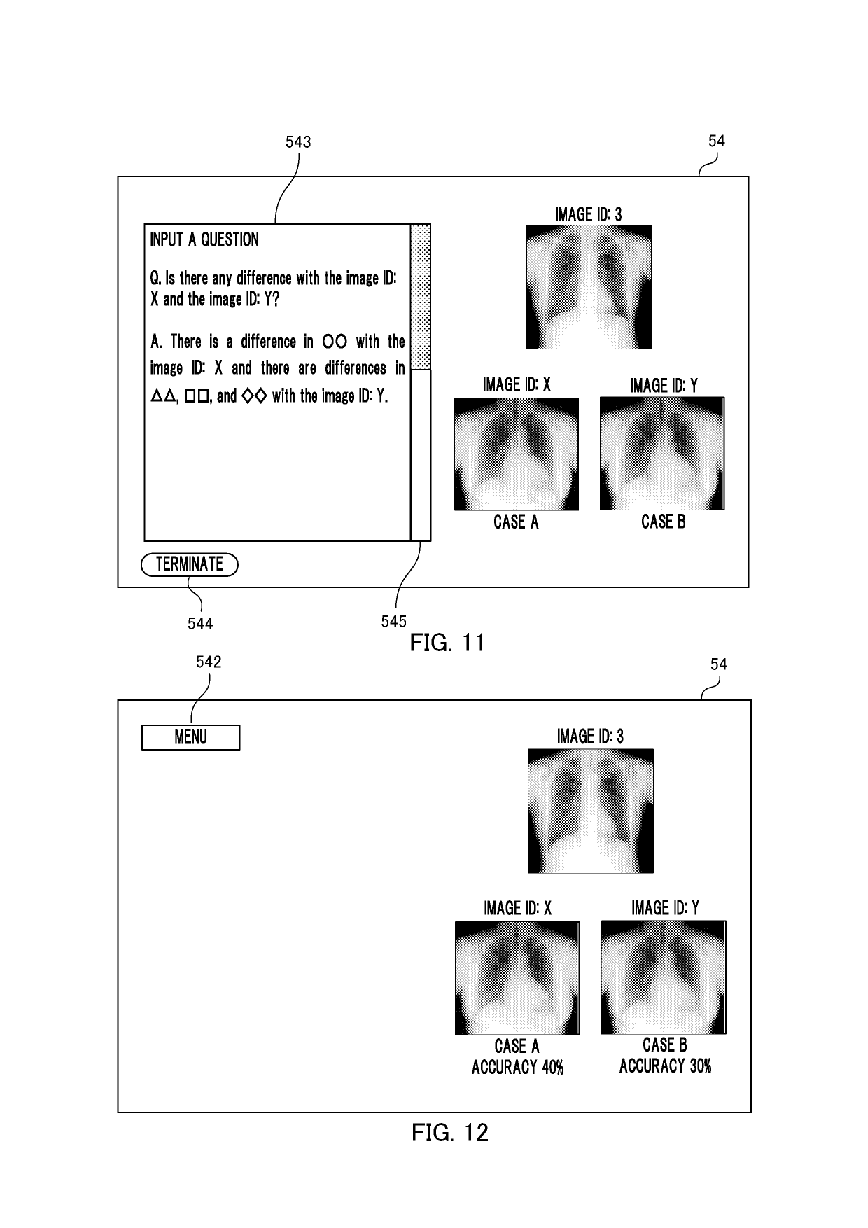

In a case where it is desired to narrow down the case candidates for the analysis images of the image ID: X and the image ID: Y which have a relatively high accuracy, a question to compare a difference(s) or the like may be inputted to narrow down the case candidates, for example, as illustrated in FIG. 11. Further, with respect to the diaphragm, a question(s) for comparing the rates of change of the lung field area and/or increases or decreases in the lung field area may also be inputted in addition to the question about the movement of the diaphragm to narrow down the case candidates.

In this case, the outputter 513 extracts a difference(s) between the analysis images of the image ID: X and the image ID: Y and the analysis image of the image ID: 3 from the analysis images of the image ID: X and the image ID: Y, and displays a response in the form of a text regarding the extracted difference(s) in the dialogue region 543 as illustrated in FIG. 11. At this time, the controller 51 may cause only the analysis images of the image ID: X and the image ID: Y to be analysis images that are displayed simultaneously with the analysis image of the image ID: 3 on the display 54 so that the medical doctor can easily visually compare these images.

In a case where the dialogue is repeated and all the dialogues cannot be displayed in the dialogue region 543, the controller 51 causes a scroll bar 545 to be displayed beside the dialogue region 543 as illustrated in FIG. 11 so that all the dialogues can be checked by operating the scroll bar 545.

The medical doctor can narrow down the case candidates for the dynamic image to be processed by repeating the dialogue with the client terminal 50, and can perform diagnosis with a higher accuracy. In the case of the present embodiment, for example, diaphragmatic eventration can be diagnosed with a high accuracy by the above-described dialogue, and in this case, the client terminal 50 displays the diagnostic result of “diaphragmatic eventration” in the dialogue region 543. That is, the client terminal 50 can assist the medical doctor in diagnosis work for the dynamic image to be processed.

Further, with respect to the narrowed-down case candidates, the controller 51 may cause a treatment plan, the next necessary inspection, and the like in a similar case to be displayed on the display 54 by retrieving and extracting data in the image management apparatus 40, for example. As a result, it is possible to assist a medical doctor not only in diagnosis of a dynamic image but also in his/her medical work going forward.

As described above, the client terminal 50 includes the display 54, the dialogue unit 512, and the outputter 513. The display 54 displays an analysis image for a dynamic image obtained by irradiating the patient M as a subject with radiation. The dialogue unit 512 executes dialogue including reception of an input of a question about the analysis image to be processed and an output of a response to the inputted question. The outputter 513 outputs case information for the dynamic image to be processed, based on the analysis image to be processed and the content of the executed dialogue.

The dialogue unit 512 and the outputter 513 output case information for the dynamic image to be processed, based on the dialogue, that is, output information for assisting a medical doctor in diagnosis. For this reason, it is possible to assist a medical doctor in his/her diagnosis-related work for a dynamic image to be processed, and to achieve improved efficiency in the work. As a result, the medical doctor can perform diagnosis with a higher accuracy with respect to a dynamic image to be processed.

### Variation 1

In the above-described embodiment, it is configured such that for a dynamic image to be processed, the controller 51 repeats dialogue with the medical doctor from the beginning to narrow down case candidates. In contrast, in the present variation, it is configured such that for a dynamic image to be processed, the controller 51 first narrows down case candidates, and then conducts dialogue with a medical doctor.

For example, in a case where accuracies of case candidates with respect to a dynamic image to be processed are high to some extent, it is possible to perform diagnosis efficiently by the controller 51 first narrowing down case candidates and then checking the case candidates and outputting final case information through dialogue with a medical doctor.

In the case of the present variation, the controller 51 further includes a narrowing-down unit 514 (see FIG. 5) that narrows down case candidates. For example, the narrowing-down unit 514 narrows down, based on the analysis image of a dynamic image to be processed, analysis images similar to the analysis image as analysis image candidates from the first retrieval data. In addition, the narrowing-down unit 514 acquires, as a dynamic image candidate(s) and a case candidate(s), a dynamic image(s) and a case(s) that have an analysis image candidate(s).

Here, the narrowing-down unit 514 is configured to include a learner and an extractor in the same manner as the outputter 513 described above. Here, the extractor retrieves and extracts a similar analysis image(s) as an analysis image candidate(s) from the first retrieval data by using a learning model with an analysis image as an input item.

In addition, the narrowing-down unit 514 also obtains the accuracy of each case candidate with respect to the dynamic image to be processed, for example, based on the similarity to the analysis image of the dynamic image to be processed, or the like.

In the case of the present variation, the narrowing-down unit 514 displays, together with the displayed analysis image of the image ID: 3, the analysis images of the image ID: X and ID: Y, which are the narrowed-down analysis image candidates, as well as the cases thereof with the accuracies thereof on the display 54 as illustrated in FIG. 12, for example.

The subsequent processing is the same as that in the above-described embodiment, and when an input operation item for starting an input of a question is selected from the menu 542 illustrated in FIG. 12, the dialogue region 543 is displayed on the display 54, and the controller 51 repeats dialogue with the medical doctor to check a case candidate(s) and output the final case information.

In this way, the controller 51 outputs case information for a dynamic image to be processed based on narrowing-down and dialogue, and thus, it is possible to assist a medical doctor in his/her diagnosis-related work for a dynamic image to be processed, and to achieve improved efficiency in the work.

### Variation 2

The above-described embodiment and Variation 1 represent cases in which dynamic analysis of a dynamic image can be performed and an analysis result and an analysis image can be acquired. However, in a case where dynamic analysis of a dynamic image cannot be performed and no analysis result and no analysis image can be acquired, that is, in a case where no analysis image for a dynamic image exists, the client terminal 50 assists work by performing the following processing.

Specifically, the display 54 displays not an analysis image, but a dynamic image. In addition, the controller 51 (the dialogue unit 512) executes dialogue including reception of an input of a question about the displayed dynamic image and an output of a response to the inputted question. In addition, the controller 51 (the outputter 513) outputs information on an imaging condition(s) for the dynamic image based on the displayed dynamic image and the content of the executed dialogue.

In the case of the present variation, the storage 52 further stores second retrieval data including a plurality of dynamic images, and an imaging condition(s) and an imaging condition(s) for re-imaging for each of the plurality of dynamic images, when no analysis result can be acquired. The second retrieval data is retrieved by the outputter 513 based on the dynamic image to be processed and the content of the executed dialogue, and a dynamic image(s) similar to the dynamic image to be processed is extracted.

The outputter 513 acquires an imaging condition(s) with the extracted dynamic image and imaging condition(s) for re-imaging, and displays a dynamic image(s) similar to the dynamic image to be processed, as well as the imaging condition(s) and imaging condition(s) for re-imaging on the display 54, with the dynamic image. In addition, the second retrieval data may include information on an imaging failure(s) in addition to the imaging condition(s), and the information may be used for retrieval and extraction or may be displayed on the display 54.

In the present variation, at the outputter 513, the learner generates a learning model by machine learning of the second retrieval data. In addition, the extractor retrieves and extracts a dynamic image(s) similar to the dynamic image to be processed, an imaging condition(s), and an imaging condition(s) for re-imaging with the dynamic image(s) from the second retrieval data by using the learning model with the dynamic image and the content of the dialogue as input items.

When the outputter 513 displays the imaging condition(s) and the imaging condition(s) for re-imaging with the dynamic image(s) similar to the dynamic image to be processed on the display 54, the outputter 513 displays the imaging condition(s) and the imaging condition(s) for re-imaging with the dynamic image(s) similar to the dynamic image to be processed in a displaying form that clarifies the difference(s) between the imaging condition(s) and the imaging condition(s) for re-imaging (for example, displaying in red, inverted displaying, blinking displaying, or the like). As described above, the imaging conditions include conditions such as an imaging posture of the patient M and the body state (respiratory state or the like) of the patient M.

In the present variation as well, the dialogue is repeated in the same manner as in the above-described embodiment, whereby the imaging condition(s) for re-imaging is/are retrieved and extracted for a dynamic image when no analysis result can be acquired and is/are displayed on the display 54. As a result, it is possible to assist a medical doctor or an imaging technician in his/her imaging work for a dynamic image, and to achieve improved efficiency in the work.

### Other Variation

In the above-described embodiment and Variation 1, the controller 51 of the client terminal 50 includes the dialogue unit 512, the outputter 513, and the narrowing-down unit 514, but for example, the controller 31 of the radiographic image analysis apparatus 30 may include the dialogue unit 512, the outputter 513, and the narrowing-down unit 514. In this case, the dialogue processing, the output processing, and the narrowing-down processing themselves are performed by the controller 31, but the displaying accompanying the dialogue processing, the output processing, and the narrowing-down processing is performed on the display 54 by the client terminal 50 (the controller 51) via the communication network N.

Any of the embodiment and variations described above is only illustration of examples for carrying out the present invention, and the technical scope of the present invention shall not be construed limitedly thereby. That is, the present invention can be carried out in various forms without departing from the gist or the main features thereof.

Although embodiments of the present invention have been described and illustrated in detail, the disclosed embodiments are made for purpose of illustration and example only and not limitation. The scope of the present invention should be interpreted by terms of the appended claims.

What is claimed is:

1. A work assistance apparatus, comprising:
a display that displays an analysis image for a dynamic image to be processed, the dynamic image to be processed having been obtained by irradiating a subject with radiation; and
at least one hardware processor, wherein
the at least one hardware processor
executes dialogue including reception of an input of a question about the displayed analysis image and an output of a response to the inputted question, and
outputs case information corresponding to the dynamic image to be processed, based on the displayed analysis image and a content of the executed dialogue.

2. The work assistance apparatus according to claim 1, wherein
the at least one hardware processor outputs a plurality of case candidates as the case information.

3. The work assistance apparatus according to claim 2, wherein
the at least one hardware processor outputs the plurality of case candidates in order of accuracy with respect to the dynamic image to be processed.

4. The work assistance apparatus according to claim 1, wherein
the at least one hardware processor performs the dialogue limited to a predetermined number of times and outputs the case information based on the displayed analysis image and the content of the dialogue that has been executed the predetermined number of times.

5. The work assistance apparatus according to claim 1, wherein
the reception includes a template for receiving the input of the question.

6. The work assistance apparatus according to claim 2, further comprising a storage that stores data including a plurality of dynamic images and a definite diagnosis result of a case based on each of the plurality of dynamic images, wherein
the at least one hardware processor outputs the plurality of case candidates from the data.

7. The work assistance apparatus according to claim 6, wherein
with respect to the plurality of dynamic images, the storage updates the data based on the definite diagnosis result of the case and a definite diagnosis result of a case based on an inspection other than an inspection using the dynamic images.

8. The work assistance apparatus according to claim 6, wherein
the at least one hardware processor generates a learning model by machine learning of the data and extracts the case information corresponding to the dynamic image to be processed from the data by using the learning model with the analysis image and the content of the dialogue as input items.

9. The work assistance apparatus according to claim 6, wherein:
the at least one hardware processor narrows down, as at least one analysis image candidate, at least one analysis image similar to the analysis image from the data based on the analysis image, and acquires at least one case in the at least one analysis image candidate as at least one case candidate,
the display displays, together with the analysis image, the at least one analysis image candidate and the at least one case candidate, and
the at least one hardware processor executes reception of an input of a question about the displayed at least one analysis image candidate and the displayed at least one case candidate, and outputs the case information based on the displayed analysis image, the displayed at least one analysis image candidate, the displayed at least one case candidate, and the content of the executed dialogue.

10. The work assistance apparatus according to claim 1, wherein:
in a case where the analysis image for the dynamic image to be processed does not exist,
the display displays the dynamic image to be processed, and
the at least one hardware processor executes reception of an input of a question about the displayed dynamic image to be processed, and outputs information on an imaging condition for the dynamic image based on the displayed dynamic image to be processed and the content of the executed dialogue.

11. A non-transitory computer-readable recording medium storing a work assistance program that causes a computer of a work assistance apparatus to execute, the work assistance apparatus being a work assistance apparatus that assists in case diagnosis of a dynamic image to be processed, the dynamic image to be processed having been obtained by irradiating a subject with radiation:
displaying an analysis image for the dynamic image to be processed;
executing dialogue including reception of an input of a question about the displayed analysis image and an output of a response to the inputted question; and
outputting case information corresponding to the dynamic image to be processed, based on the displayed analysis image and a content of the executed dialogue.
